(19) United States
(12) Patent Application Publication
(10) Pub. No.: US 2025/0229128 A1
(43) Pub. Date: Jul. 17, 2025

(54) SYSTEM AND METHOD FOR TEACHING GOLF

(71) Applicants: JUNG CHAN AHN, Seoul (KR); BOKHEE YU, Seoul (KR)

(72) Inventors: JUNG CHAN AHN, Seoul (KR); BOKHEE YU, Seoul (KR)

(21) Appl. No.: 19/013,316

(22) Filed: Jan. 8, 2025

(30) Foreign Application Priority Data
Jan. 12, 2024 (KR) 10-2024-0005681

(51) Int. Cl. A63B 24/00; A63B 71/06; A63B 102/32

(52) U.S. Cl. CPC A63B 24/0006; A63B 71/0622; A63B 2024/0068; A63B 2071/0647; A63B 2102/32; A63B 2214/00; A63B 2220/05; A63B 2220/30; A63B 2220/807; A63B 2220/89; A63B 2225/20

(57) ABSTRACT

A method for performing golf lessons on a server includes receiving information on the gender, age, and body type of the golfer and setting a lesson goal for the golfer, analyzing the golfer's swing motion acquired from the server and analyzing the club face control and lever system of the golfer, analyzing the path control of the club according to the golfer's swing, analyzing the control accuracy of the impact of hitting a golf ball and the flight of the golf ball, analyzing the consistency between the golfer's golf swing and the flight of the golf ball resulting therefrom over a plurality of times, analyzing the head speed of the club and the speed of the golf ball, and determining whether the set lesson goal of the golfer has been achieved.

### CROSS REFERENCE TO RELATED APPLICATION AND CLAIM OF PRIORITY

This application claims the benefit under 35 USC § 119 of Korean Patent Application No. 10-2024-0005681 filed on Jan. 12, 2024, in the Korean Intellectual Property Office, the entire disclosure of which is incorporated herein by reference for all purposes.

### BACKGROUND

### 1. Technical Field

The present disclosure relates to a golf instruction method and system, and more specifically, to a method for efficiently and systematically coaching the swing of a golf trainee from the perspective of teaching golf.

### 2. Background Art

Golf, previously known as an aristocratic sport, is expanding into a popular sport in Korea. According to one survey, the number of golfers in Korea was 5.64 million as of 2021, surpassing Japan's 5.6 million.

As the demand for golf increases, the golf market size has also become a fairly large market, reaching KRW 6.9599 trillion (excluding caddie fees) as of 2021.

In popularizing golf, numerous new golfers, aspiring professional golfers, and professional teachers (lesson professionals) who teach them played a significant role.

In most cases, they go through a beginner's course in golf by receiving coaching on golf basics and swing from professional teachers for several months at an indoor practice range, screen practice range, or indoor practice range, and then going out to the actual field and playing golf games.

However, since the teaching methods of each professional teachers are different and the physical structure and level of practice of each golfer receiving lessons are different, there is a lack of a unified teaching method or integrated education system for systematic golf instruction.

Therefore, in this disclosure, a golf teaching method that can be applied to golf lessons by most professional teachers is disclosed.

### SUMMARY

The present disclosure is intended to solve the problems of the above-mentioned prior art, and discloses an efficient and systematic teaching method for golf.

One embodiment of the present invention relates to a method for performing golf lessons on a server, the method including: a first step of receiving information on the gender, age, and body type of the golfer and setting a lesson goal for the golfer; a second step of analyzing the golfer's swing motion obtained from the server and analyzing the club face control and lever system of the golfer; a third step of analyzing the path control of the club according to the golfer's swing; a fourth step of analyzing the control accuracy of the impact of hitting a golf ball and the flight of the golf ball; a fifth step of analyzing the consistency between the golfer's golf swing and the flight of the golf ball resulting therefrom over a plurality of times; a sixth step of analyzing the head speed of the club and the speed of the golf ball; and a seventh step of determining whether the golfer has achieved the set lesson goal.

In addition, the server analyzes the consistency between the golf swing and the flight of the golf ball accordingly, and if it determines that the predetermined condition is not satisfied, it moves to the third step of analyzing the path control of the club.

In addition, the server analyzes the club head speed and the golf ball speed, and if it determines that they do not meet the predetermined condition, it moves to the second step of analyzing the club face control and lever system and performs re-learning.

In addition, the server determines whether the golfer's set lesson goal has been achieved, and if it is determined that the lesson goal has been achieved, sets a lesson goal higher than the previously set lesson goal.

In addition, the steps 1 to 7 are characterized in that each step includes sub-steps, and goals can be set for each sub-step.

Another embodiment of the present invention relates to a golf teaching system, which includes: a terminal for receiving information on a golfer's gender, age, and body type, and for capturing a swing motion of the golfer; and a server, wherein the server includes: a golf motion analysis unit for analyzing a club face control and lever system, a path control of a club according to a swing, control accuracy of an impact when hitting a golf ball and the flight of a golf ball, consistency between a plurality of golf swings of the golfer and the flight of the golf ball, a head speed of the club, and a speed of the golf ball; a goal achievement determination unit for receiving a lesson goal of the golfer and determining whether the lesson goal of the golfer has been achieved; and a teaching saving unit for saving the teaching information of the golfer and creating a database.

In addition, the goal achievement determination unit is characterized in that it analyzes the consistency between the golf swing and the flight of the golf ball accordingly, and if it determines that the consistency does not meet a predetermined condition, it is set to request the golfer's swing again to reanalyze the path control of the club.

In addition, the goal achievement determination unit is characterized in that, if it determines that the head speed of the club and the speed of the golf ball do not meet the predetermined condition by analyzing them, it is set to request the golfer's swing again to reanalyze the club face control and lever system.

In addition, the goal achievement determination unit is characterized in that it determines, in the server, whether the golfer's set lesson goal has been achieved, and, if it is determined that the lesson goal has been achieved, sets a higher lesson goal than a previously set lesson goal.

In addition, the goal achievement determination unit is characterized by setting sub-goals for each of the club face control and lever system, club path control according to swing, control accuracy of impact when hitting a golf ball and flight of the golf ball, consistency between a plurality of golf swings of the golfer and the flight of the golf ball, club head speed, and golf ball speed, and determining whether the set sub-goals have been achieved.

The present disclosure relates to a golf teaching method and system, and can provide an efficient and systematic teaching method for golf.

In addition, as a golfer, you can continuously receive feedback on your swing posture, and set sub-goals for each stage to check whether the goal has been achieved.

In addition, as a professional teacher, by adopting the systematic instruction method of the present invention, you can integrate the teaching methods of each professional teacher, thereby maintaining consistency in instruction among professional teachers.

In addition, instead of the professional teacher visually checking the golfer's movements, the movement of the golf club, and the flight of the golf ball, you can analyze the error from the guideline through images captured through a terminal and a golf ball tracking radar, etc., thereby correcting the posture and golf club control, thereby providing objective feedback to the golfer.

### BRIEF DESCRIPTION OF THE DRAWINGS

FIG. 1 is a conceptual diagram describing a golf teaching method according to an embodiment of the present invention.

FIG. 2 is a diagram describing a golf teaching process according to an embodiment of the present invention.

FIGS. 3 to 5 are diagrams describing each step of a golf teaching process according to an embodiment of the present invention.

FIG. 6 is a diagram describing a golf lesson goal setting step according to an embodiment of the present invention.

FIG. 7 is a diagram describing a club face control and lever system according to an embodiment of the present invention.

FIG. 8 is a diagram describing a club path control according to an embodiment of the present invention.

FIG. 9 is a diagram describing an alignment of an impact according to an embodiment of the present invention.

FIG. 10 is a diagram describing a swing according to an embodiment of the present invention and consistency between the flight of a golf ball according to the swing.

FIG. 11 is a diagram describing a smash factor that analyzes a club head speed and a golf ball speed according to an embodiment of the present invention.

FIG. 12 is a diagram describing a goal achievement check according to an embodiment of the present invention.

FIG. 13 is a diagram conceptually describing the structure of a server according to an embodiment of the present invention.

### DETAILED DESCRIPTION OF THE INVENTION

Hereinafter, with reference to the attached drawings, embodiments of the present invention are described in detail so that those with ordinary skill in the art can easily practice the present invention. However, the present invention may be implemented in various different forms and is not limited to the embodiments described herein. In addition, in order to clearly describe the present invention in the drawings, parts that are not related to the description are omitted, and similar parts are assigned similar drawing reference numerals throughout the specification.

Hereinafter, various embodiments of the present disclosure will be described with reference to the accompanying drawings. However, this is not intended to limit the technology described in the present disclosure to specific embodiments, but should be understood to include various modifications, equivalents, and/or alternatives of the embodiments of the present disclosure. In connection with the description of the drawings, similar reference numerals may be used for similar components.

In the present disclosure, expressions such as “has,” “can have,” “includes,” or “may include” indicate the presence of a corresponding feature (e.g., a component such as a number, function, operation, or part), and do not exclude the presence of additional features.

In this disclosure, the expressions “A or B,” “at least one of A and/or B,” or “one or more of A or/and B” can include all possible combinations of the listed items. For example, “A or B,” “at least one of A and B,” or “at least one of A or B” can all refer to (1) including at least one A, (2) including at least one B, or (3) including both at least one A and at least one B.

The expressions “first,” “second,” “first,” or “second,” etc., used in this disclosure can describe various components, regardless of order and/or importance, and are only used to distinguish one component from another, but do not limit the components. For example, a first user device and a second user device can represent different user devices, regardless of order or importance. For example, without departing from the scope of the rights set forth in this disclosure, a first component can be named a second component, and similarly, a second component can also be renamed as a first component.

When it is said that a component (e.g., a first component) is “(operatively or communicatively) coupled with/to” or “connected to” another component (e.g., a second component), it should be understood that said component is directly coupled to/to the other component, or can be connected via another component (e.g., a third component). On the other hand, when it is said that a component (e.g., a first component) is “directly coupled to” or “directly connected to” another component (e.g., a second component), it should be understood that no other component (e.g., a third component) exists between said component and said other component.

The expression “configured to” as used in this disclosure can be used interchangeably with, for example, “suitable for,” “having the capacity to,” “designed to,” “adapted to,” “made to,” or “capable of.” The term “configured to” does not necessarily mean only something that is “specifically designed to” in terms of hardware. Instead, in some contexts, the expression “a device configured to” can mean that the device is “capable of” doing something together with other devices or components. For example, the phrase “a processor configured (or set) to perform A, B, and C” can mean a dedicated processor (e.g., an embedded processor) to perform those operations, or a generic-purpose processor (e.g., a CPU or an application processor) that can perform those operations by executing one or more software programs stored in a memory device.

The terms used in this disclosure are only used to describe specific embodiments and may not be intended to limit the scope of other embodiments. The singular expression may include the plural expression unless the context clearly indicates otherwise. The terms used herein, including technical or scientific terms, may have the same meaning as commonly understood by a person having ordinary skill in the art described in this disclosure. Among the terms used in this disclosure, terms defined in general dictionaries may be interpreted as having the same or similar meaning as the meaning they have in the context of the related technology, and shall not be interpreted in an ideal or excessively formal meaning unless explicitly defined in this disclosure. In some cases, even if a term is defined in this disclosure, it cannot be interpreted to exclude embodiments of the present disclosure.

An electronic device according to various embodiments of the present disclosure may include, for example, at least one of a smartphone, a tablet personal computer (PC), a mobile phone, a video phone, an e-book reader, a desktop personal computer (PC), a laptop personal computer (PC), a netbook computer, a workstation, a server, a personal digital assistant (PDA), a portable multimedia player (PMP), an MP3 player, a mobile medical device, a camera, a wearable device, or a radar. According to various embodiments, the wearable device may include at least one of an accessory type (e.g., a watch, a ring, a bracelet, an anklet, a necklace, glasses, a contact lens, or a head-mounted device (HMD)), a fabric or clothing-integrated type (e.g., an electronic garment), a body-attached type (e.g., a skin pad or a tattoo), or a bio-implantable type (e.g., an implantable circuit).

In the present invention, “golfer” refers to a user who swings a golf club and hits a golf ball, and refers to a user who enjoys a golf game or takes golf lessons. In the present invention, amateurs, semi-professionals, and professionals are broadly referred to as golfers.

In the present invention, “club” refers to a golf club handled by a golfer, for example, a golf club such as a driver, wood, hybrid/utility, iron, wedge, and putter.

In the present invention, “swing” refers to a series of processes for hitting a golf ball using a golf club. A swing refers to a series of movements such as address, take away, back swing, down swing, impact, and finish being performed continuously.

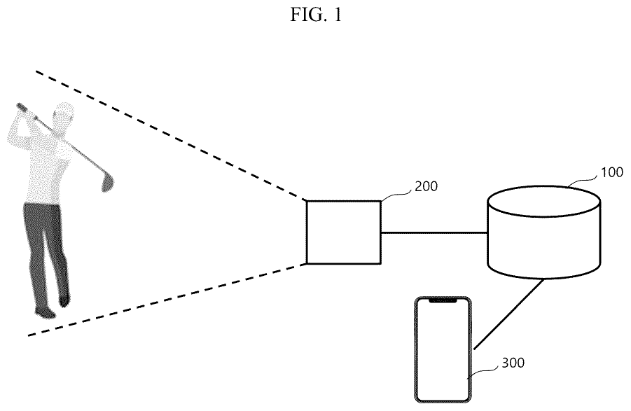

FIG. 1 is a conceptual diagram describing a golf teaching method according to an embodiment of the present invention.

A golf teaching method according to an embodiment of the present invention is performed by capturing a golfer's swing motion, a golf club's movement, and a golf ball's flight at a terminal (200), transmitting the images to a server (100) connected to the terminal (200) via a network, and analyzing the golfer's swing motion and the golf ball's flight at the server (100) to provide feedback.

A server (100) according to an embodiment of the present invention analyzes the training information of a golfer and executes a program designed as an algorithm to improve the golfer's golf skills.

A terminal (200) according to an embodiment of the present invention receives information about a golfer (e.g., gender, age, body type) and captures the golfer's swing motion, the movement of a golf club, and the flight of a golf ball. A terminal (200) according to an embodiment of the present invention includes a camera sensor and a radar, and captures the golfer's swing motion, the movement of a golf club, and the flight of a golf ball and transmits the photographs to the server (100).

In addition, the terminal (200) according to an embodiment of the present invention can receive information identifying the golfer and determine who the golfer is to be image-captured.

The teaching system according to an embodiment of the present invention may further include a user terminal (300), and the user terminal (300) may be a device that displays feedback information on the golfer's learning.

The user terminal (300) according to an embodiment of the present invention may display a swing image captured by the terminal (200) so that the golfer can check it, or display content analyzed by the server (100) so that the golfer can check it.

The user terminal (300) according to an embodiment of the present invention may be a terminal that allows a golfer or a professional teacher to set a lesson goal or check a teaching history, and for example, a smartphone, an AI phone, a tablet, a laptop, a desktop, a MAC, etc. may correspond to the user terminal (300).

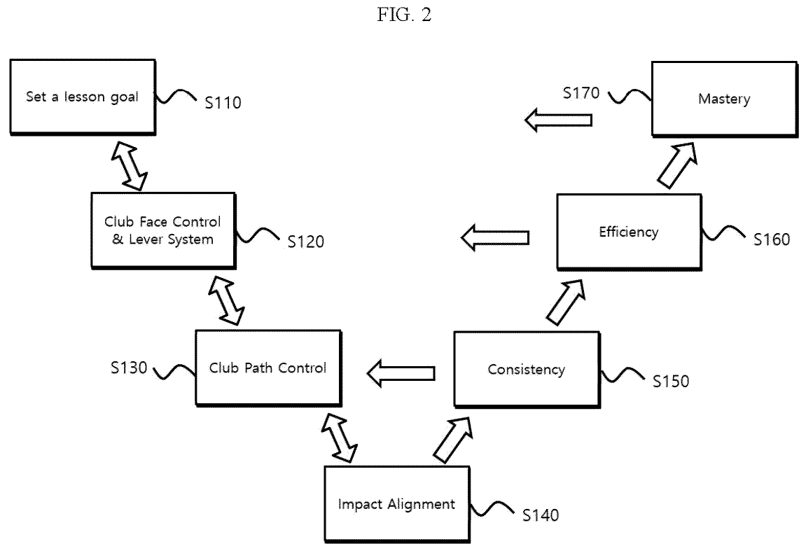

FIG. 2 is a drawing describing a golf teaching process according to an embodiment of the present invention.

As shown in FIG. 2, the golf teaching process according to an embodiment of the present invention proceeds according to a series of processes.

First, the golfer can set the goal of the lesson before taking the golf lessons (S110). The server can set the level of goal achievement according to the golfer's lesson goal.

After that, the server analyzes the acquired swing motion of the golfer to analyze the golfer's club face control and lever system (S120). At this time, if the server determines that the goal is not achieved in this process, it can receive information on resetting the golfer's lesson goal (S110).

After that, the server analyzes the path control of the club according to the golfer's swing (S130). At this time, if the server determines that the goal is not achieved in this process, it can receive reanalysis (S120) information on the golfer's club face control and lever system.

After that, the server analyzes the control accuracy of the impact hitting the golf ball and the flight of the golf ball (S140). At this time, if the server determines that the goal is not achieved in this process, it can receive reanalysis (S130) information on the path control of the club according to the golfer's swing.

After that, the server analyzes the consistency between the golfer's swing and the flight of the golf ball according to the swing for a plurality of times (S150). At this time, if the server determines that the goal is not achieved in this process, it can receive reanalysis (S130) information on the path control of the club according to the golfer's swing.

After that, the server measures efficiency (S160). According to an embodiment of the present invention, the server analyzes the smash factor (the head speed of the club and the speed of the golf ball) as an example for measuring efficiency. At this time, if the server determines that the goal is not achieved in this process, it can receive reanalysis (S120) information on the golfer's club face control and lever system.

Afterwards, the server determines whether the previously set goal (S110) has been achieved (S170). If it is determined that the goal has been achieved, the golfer's lesson goal can be adjusted upwards so that the golfer can perform a teaching course with a higher difficulty level. Conversely, if it is determined that the goal has not been achieved, the golfer's lesson goal can be adjusted downwards so that the golfer can perform a taking course with a lower difficulty level or the same difficulty level.

By continuously performing the above series of processes, the golfer can receive continuous feedback and objective analysis information on the swing motion, which has the advantageous effect of improving golf skills.

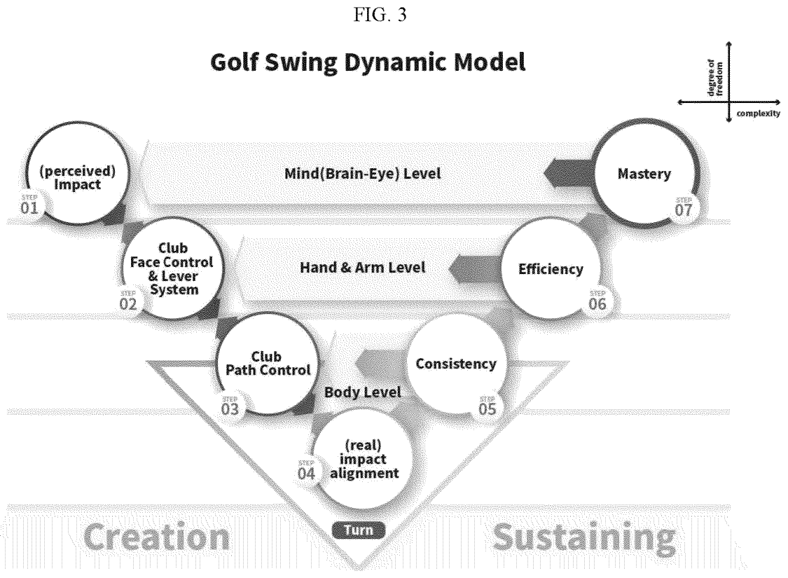

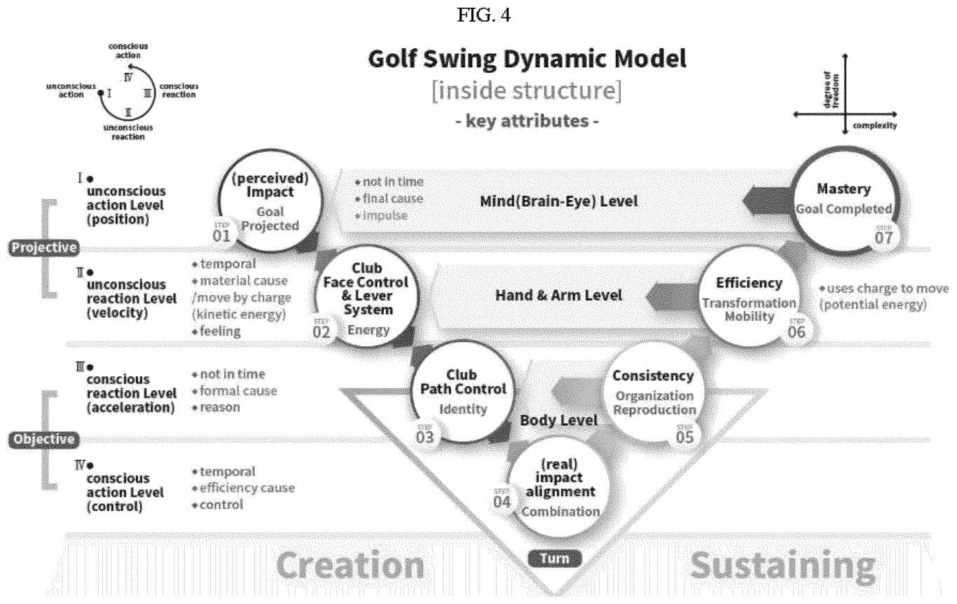

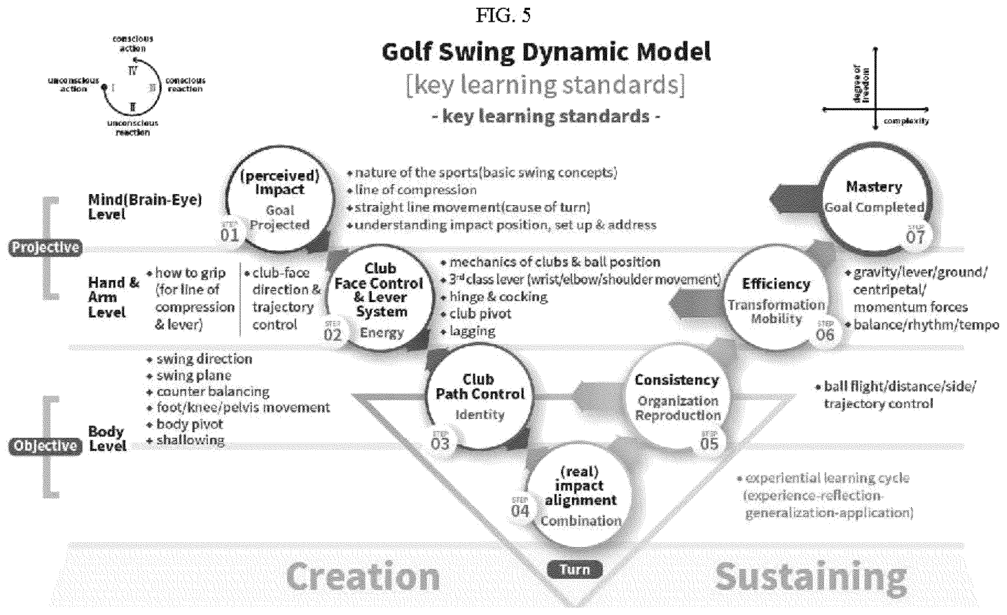

FIGS. 3 to 5 are drawings describing each stage of a golf teaching process according to an embodiment of the present invention.

As shown in FIG. 3, a series of teaching processes of the present invention are distributed on multiple levels.

First, the first stage of goal setting and the seventh stage of the goal achievement determining stage are a kind of mind (Brain-Eye) stage, which is a stage for setting a goal that the golfer wants to achieve through the teaching process. For example, as an amateur golfer who hits a tee shot from a white tee, a goal of becoming a bogey player (total of 90 strokes) and the impact control required for the goal can be set.

The second stage of club face control and lever system and the sixth stage of analyzing the club head speed and golf ball speed are a kind of hand and arm training stage, which is a practice stage for grip movement of holding the club, lever system, etc.

After that, the third stage of club path control and the fifth stage of consistency between golfers' golf swings are the golfer's body stage, which constitutes one stage following the mind stage and the hand and arm training stage.

Finally, the fourth stage of the impact control accuracy is a stage that constitutes a turning point of the above stages, and is a stage that occurs when each level continuously gives and receives feedback on posture correction.

The first to fourth stages are stages that can be mutually repeated or returned to each other, and the fourth to seventh stages are organically connected as a process of moving to a designated stage. Each stage will be described in more detail below.

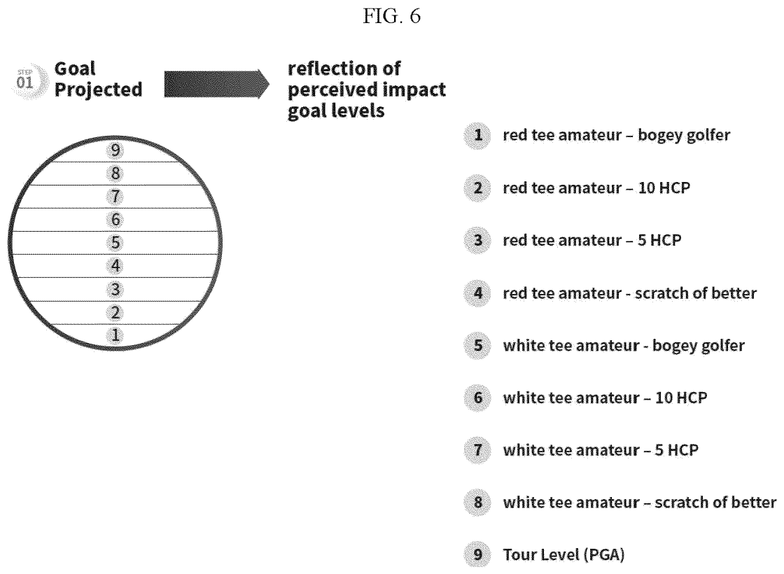

FIG. 6 is a drawing describing a golf lesson goal setting step according to an embodiment of the present invention.

As shown in FIG. 6, the first step of goal setting is a step of setting a goal of skill (number of strokes) that a golfer wants to achieve through lesson. As shown in FIG. 6, the first to seventh steps of the golf teaching process of the present invention can be composed of each sub-step, and golf teaching can be performed in a manner of upwardly adjusting the goal while achieving these sub-steps.

The first step of goal setting according to an embodiment of the present invention is a mind level, and is a step of practicing basic knowledge such as understanding of basic swing movements, movement according to trajectory, understanding of impact, posture setup, and address.

Lesson goals for skill can be set based on various criteria. Tee shot locations such as red tee (ladies tee), white tee, etc., indicating the distance to tee off, can be set based on criteria such as bogey player (90 strokes), 10 handicap (HCP), 5 handicap, etc., indicating the number of strokes. Or, professional levels such as amateur, tour pro (PGA), etc. can be set as one goal.

As a sub-level of the first level, if the goal is set to level 1-1 red tee amateur (bogey player), and then achieved, the next goal can be level 1-2 red tee amateur (10 handicap).

The server can set each sub-level to be progressively higher (step by step), but in some cases, depending on the golfer's lesson results, some steps can be omitted and set to a higher level.

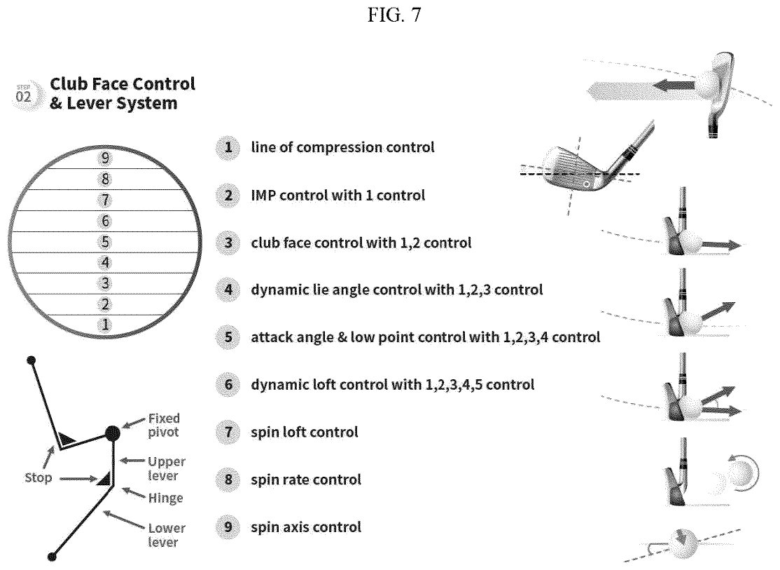

FIG. 7 is a drawing describing a club face control and lever system according to an embodiment of the present invention.

As shown in FIG. 7, the step of analyzing the club face control and lever system is a step regarding the hand and arm of the golfer, and analyzes the method of holding the grip, the mechanism for the position of the club and the golf ball, and the control for the direction of the club face and the swing trajectory.

In addition, as a lever system, the server analyzes the posture in which the connection of the three parts of the golfer's club swing, namely the wrist, elbow, and shoulder, forms one lever.

In addition, the server can analyze the hinge motion in which the golfer's hands are clasped together and the fingers are bent toward the back of each hand, and the coking posture in which the thumbs are bent upward when the hands are clasped together.

In addition, the server can analyze the pivoting motion, which indicates the change in direction of the club, and the lagging motion, which causes the club head to follow the downswing.

The analysis of the golfer's motion can be performed by comparing and analyzing the error between the guidelines set for the server and the golfer's motion, and the server can determine that the practice is sufficient if the error is smaller.

In the second stage, various sub-level goals can be set. For example, the goals can be set as sub-levels such as understanding the trajectory (level 2-1), club face control (level 2-3), and rotation axis control (level 2-9) with the easiest difficulty.

In the present invention, the sub-levels of the process of steps 1 to 7 can be set as different goals, and for example, if the sub-level of step 1-2 is set in step 1 (mind), it is not necessarily set as the sub-level of step 2-2 in the second stage, but can be set as a different sub-level depending on the golfer's practice results.

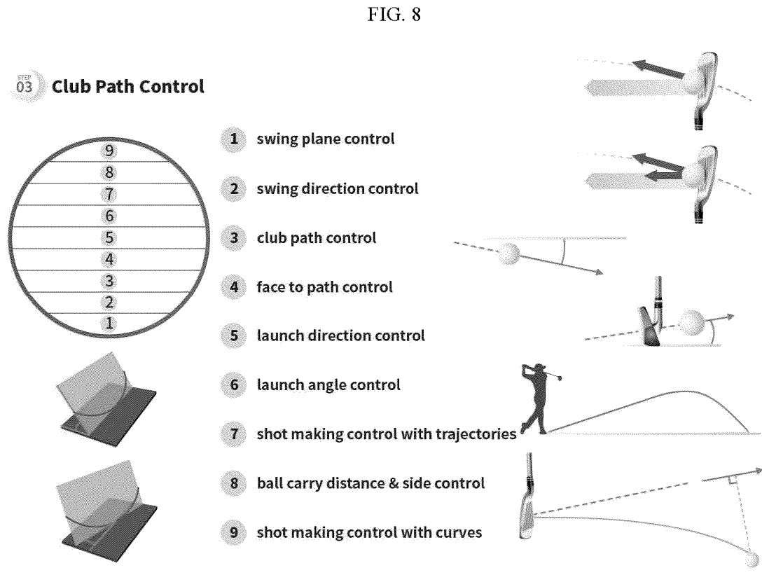

FIG. 8 is a drawing describing a club path control according to an embodiment of the present invention.

As shown in FIG. 8, as a third step of analyzing the body movement of a golfer, the server analyzes the club path control, including the swing direction, the swing plane, counter balancing indicating the balance of the body with respect to the club, the movement of the feet, knees and pelvis, the pivot of the body, and the shallowing motion in which the club falls back or lies down.

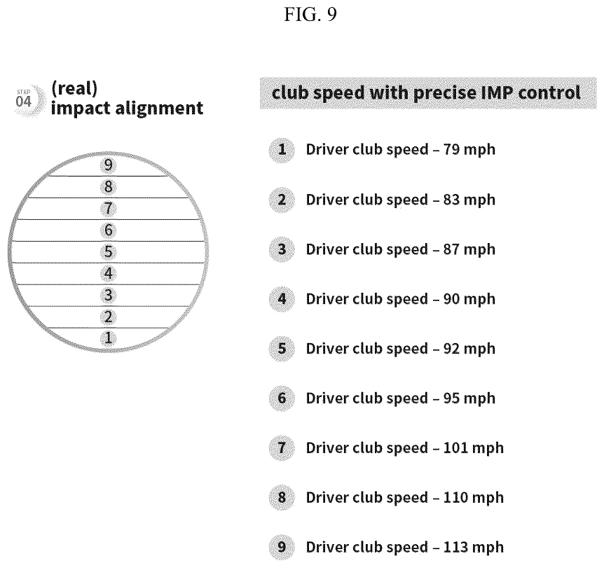

FIG. 9 is a drawing describing the alignment of impact according to an embodiment of the present invention.

As shown in FIG. 9, the fourth stage of impact has a meaning as a turning point among the seven stages, and the server analyzes the ability to adjust (align) the exact impact that can create the flight of the golf ball with the desired quality of the golfer. In the case of impact alignment, it refers to the ability to implement the impact that the golfer perceives (perceived impact) for the flight of the golf ball with the desired quality of the golfer into the actual impact (real impact).

The golfer can set various sub-stage goals, such as 79 mph (stage 4-1) for the driver club speed, 92 mph (stage 4-5), etc., and the server can determine whether these goals are achieved.

Stages 1 to 4 above are meaningful as the creation stage of the motion to hit and send the golf ball, and stages 4 to 7 through impact have the meaning of maintaining the created motion.

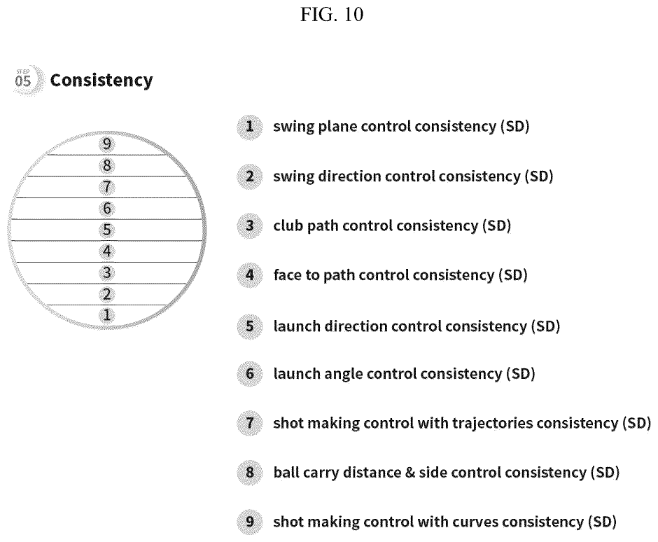

FIG. 10 is a diagram describing consistency between swings and the flight of a golf ball according to an embodiment of the present invention.

As shown in FIG. 10, the fifth step of analyzing consistency between swings analyzes consistency between swings and the flight of a golf ball according to swings at the server when a golfer swings a plurality of times.

A golfer swings a plurality of times during practice, and a server according to an embodiment of the present invention analyzes the consistency (whether or not it matches) between the ball height, distance, direction, and trajectory of each swing as an evaluation index.

For example, a server according to an embodiment of the present invention can analyze whether the club path is consistent between a plurality of swings (step 5-3) or whether the launch angle is consistent (step 5-6).

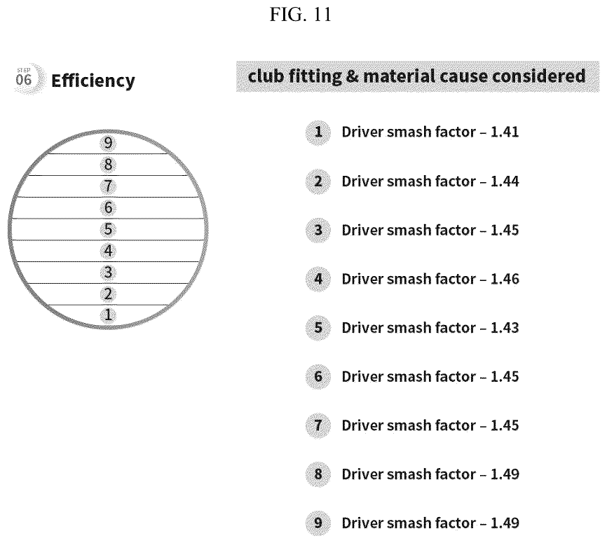

FIG. 11 is a diagram describing a smash factor that analyzes the head speed of a club and the speed of a golf ball according to an embodiment of the present invention.

As shown in FIG. 11, the server according to an embodiment of the present invention measures efficiency as the sixth step, and as a representative example of efficiency measurement, analyzes the head speed of the club and the speed of the golf ball.

The server according to an embodiment of the present invention can calculate a variable called a smash factor by analyzing the head speed information of the club and the speed of the golf ball.

The smash factor in the present invention can be calculated as shown in the following mathematical expression 1.

Smash Factor=Ball Speed/Head Speed [Expression 1]

Smash factor refers to the correlation between head speed and ball speed. Head speed and ball speed are important factors in determining distance, and are numerical values that indicate whether head speed properly transmits power to the ball. In other words, it can be interpreted that the higher the smash factor, the longer the distance of the golf ball.

For example, a server can set the smash factor of a driver to 1.44 (step 6-2) and then adjust it upward to 1.49 (step 6-8) if this condition is met.

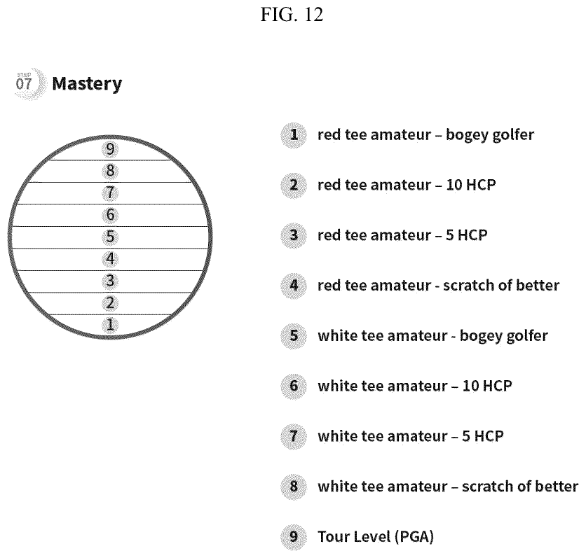

FIG. 12 is a drawing describing a goal achievement check according to an embodiment of the present invention.

A server according to an embodiment of the present invention can determine whether each goal has been achieved as a result of the golfer's practice according to the instruction in steps 1 to 6. This can be set by determining whether a lower-level goal set in advance by the server has been achieved, and setting a new upwardly adjusted goal to perform steps 1 to 7 again.

A golfer can improve his/her skills by continuously setting upwardly adjusted goals and achieving them, and as a professional teacher, he/she can perform efficient and systematic instruction by coaching based on objective data.

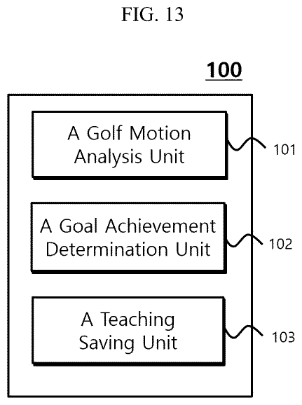

FIG. 13 is a conceptual diagram describing the structure of a server according to an embodiment of the present invention.

As described in FIG. 13, a server (100) according to an embodiment of the present invention includes a golf motion analysis unit (101), a goal achievement determination unit (102), and a teaching saving unit (103). Although not described in FIG. 13, the server (100) may further include an input/output unit, a display unit, a power supply unit, a storage unit, a communication unit, a sensor unit, etc. for smooth operation.

A golf motion analysis unit (101) according to an embodiment of the present invention analyzes the club face control and lever system, the club path control according to the swing, the control accuracy of the impact of hitting the golf ball and the flight of the golf ball, the consistency between the golfer's golf swing and the flight of the golf ball according to the golf swing over a plurality of times, the head speed of the club, and the speed of the golf ball. For this analysis, the server (100) receives the golfer's swing video and the flight trajectory of the golf ball from the terminal, and can perform an evaluation by comparing and analyzing the error between the golfer's swing motion and the flight of the golf ball according to the golf swing and the guideline.

A goal achievement determination unit (102) according to an embodiment of the present invention receives the golfer's lesson goal and determines whether the golfer's lesson goal has been achieved.

The goal achievement determination unit (102) according to one embodiment of the present invention analyzes the consistency between the golf swings and, if it determines that the predetermined conditions are not satisfied, can be set to request the golfer's swing again to reanalyze the path control of the club so that the golfer can relearn.

The goal achievement determination unit (102) according to an embodiment of the present invention analyzes the head speed of the club and the speed of the golf ball, and if it determines that the predetermined conditions are not satisfied, it can be set to request the golfer's swing again to reanalyze the club face control and lever system so that the golfer can relearn.

The goal achievement determination unit (102) according to an embodiment of the present invention can be set to determine in the server whether the golfer's set lesson goal has been achieved, and if it is determined that the lesson goal has been achieved, it can be set to set a higher lesson goal than the previously set lesson goal.

The teaching saving unit (103) according to an embodiment of the present invention can save the teaching information of the golfer and create a database. The teaching saving unit (103) according to an embodiment of the present invention can analyze the degree of skill improvement of each golfer by accumulating and storing the teaching history of each identified golfer in a database, and can provide systematic data on the movement practice that requires supplementation.

In addition, the teaching saving unit (103) according to an embodiment of the present invention does not simply record and save the teaching information of each golfer, but also provides teaching information from the teaching of another golfer at the same stage, and can create an environment that can be used as reference information. For example, if a golfer continues to lift his head during a swing motion, the teaching saving unit (103) according to an embodiment of the present invention can provide a video of another golfer who is well trained in this area and provide a reference for following.

An embodiment of the present invention may also be implemented in the form of a recording medium containing computer-executable instructions, such as program modules, that are executed by a computer. Computer-readable media can be any available media that can be accessed by a computer, and includes both volatile and nonvolatile media, removable and non-removable media. Additionally, computer-readable media can include both computer storage media and communication media. Computer storage media includes both volatile and nonvolatile, removable and non-removable media implemented in any method or technology for storage of information, such as computer-readable instructions, data structures, program modules, or other data. Communication media typically includes computer-readable instructions, data structures, program modules, or other data in a modulated data signal, such as a carrier wave, or other transport mechanism, and includes any information delivery media.

Although the method and system of the present invention have been described with respect to specific embodiments, some or all of their components or operations may be implemented using a computer system having a general hardware architecture.

The foregoing description of the present invention is for illustrative purposes, and those skilled in the art will understand that the present invention can be easily modified into other specific forms without changing the technical idea or essential features of the present invention. Therefore, it should be understood that the embodiments described above are exemplary in all respects and not limiting. For example, each component described as a single type may be implemented in a distributed manner, and likewise, components described as distributed may be implemented in a combined form.

The scope of the present invention is indicated by the claims described below rather than the detailed description above, and all changes or modifications derived from the meaning and scope of the claims and their equivalent concepts should be interpreted as being included in the scope of the present invention.

What is claimed is:

1. A method for performing golf lesson on a server, the method comprising:
receiving information on the golfer's gender, age, and body type, and setting a golfer's lesson goal;
analyzing the golfer's swing motion obtained from the server, and analyzing the golfer's club face control and lever system;
analyzing the club's path control according to the golfer's swing;
analyzing the control accuracy of the impact of hitting a golf ball and the flight of the golf ball;
analyzing the consistency between the golfer's golf swing and the flight of the golf ball according to the golf ball over a plurality of times;
analyzing the club's head speed and the speed of the golf ball; and
determining whether the golfer's set lesson goal is achieved.

2. The method of claim 1, wherein, if the server analyzes the consistency between the golf swing and the flight of the golf ball accordingly and determines that it does not satisfy a predetermined condition, it moves to a third step of analyzing the path control of the club and performs re-learning.

3. The method of claim 1, wherein, when the server analyzes the club head speed and the golf ball speed and determines that they do not meet the predetermined conditions, the method moves to the second step of analyzing the club face control and lever system.

4. The method of claim 1, wherein, in the server, it is determined whether the golfer's set lesson goal has been achieved, and if it is determined that the lesson goal has been achieved, a higher lesson goal is set than the previously set lesson goal.

5. The method of claim 1, wherein each step comprises a sub-step, and a goal can be set for each sub-step.

6. A golf teaching system comprising,
a terminal for receiving information on the gender, age, and body type of a golfer, and capturing the swing motion of the golfer, the movement of the golf club, and the flight of the ball; and
a server comprising:
a golf motion analysis unit configured for analyzing a club face control and lever system, a path control of the club according to the swing, the control accuracy of the impact of hitting the golf ball and the flight of the golf ball, the consistency between the golfer's golf swing and the flight of the golf ball over a plurality of times, the head speed of the club, and the speed of the golf ball;
a goal achievement determination unit configured for receiving the golfer's lesson goal and determining whether the golfer's lesson goal has been achieved; and
a teaching saving unit configured for saving the golfer's teaching information and creating a database.

7. The system of claim 6, wherein the goal achievement determination unit is configured to set to analyze the consistency between the golf swing and the flight of the golf ball accordingly, and if it determines that the predetermined condition is not satisfied, to request the golfer's swing again to reanalyze the path control of the club.

8. The system of claim 6, wherein, if it analyzes the club head speed and the golf ball speed and determines that the predetermined conditions are not met, it is set to request the golfer's swing again to reanalyze the club face control and lever system.

9. The system of claim 6, wherein, in the server, it judges whether the golfer's set lesson goal has been achieved, and, if it is judged that the lesson goal has been achieved, it sets a higher lesson goal than the previously set lesson goal.

10. The system of claim 6, wherein it sets sub-goals for each of the club face control and lever system, the club path control according to the swing, the control accuracy of the impact hitting the golf ball and the flight of the golf ball, the consistency between the golfer's golf swing and the flight of the golf ball according to the golf swing, the club head speed and the golf ball speed, and determines whether the set sub-goals have been achieved.
